(19) United States
(12) Patent Application Publication
(10) Pub. No.: US 2021/0322333 A1
(43) Pub. Date: Oct. 21, 2021

(54) Lignin-PLGA Biopolymers and Nanoparticles, and their Synthesis and Use

(71) Applicant: Board of Supervisors of Louisiana State University and Agricultural and Mechanical College, Baton Rouge, LA (US)

(72) Inventors: Carlos Astete, Baton Rouge, LA (US); Cristina Sabliov, Baton Rouge, LA (US)

(73) Assignee: Board of Supervisors of Louisiana State University and Agricultural and Mechanical College, Baton Rouge, LA (US)

(21) Appl. No.: 17/283,730

(22) PCT Filed: Oct. 9, 2019
(86) PCT No.: PCT/US19/55299

§ 371 Date: Apr. 8, 2021

Related U.S. Application Data
(60) Provisional application No. 62/898,266, filed on Sep. 10, 2019.
(60) Provisional application No. 62/744,233, filed on Oct. 11, 2018.

(51) Int. Cl. A61K 9/51; C08G 81/00; A61K 31/437

(52) U.S. Cl. CPC A61K 9/5153; C08G 81/00; B82Y 5/00; A61K 31/437; A61K 9/5192

(57) ABSTRACT

Amphiphilic biopolymers have been synthesized by grafting lignin onto PLGA to form graft polymers, which can then be further assembled into polymeric nanoparticles without a requirement for surfactants. The nanoparticles have a typical diameter of 75 nm. The nanoparticles may be used, for example, for drug delivery, including efficient and effective drug delivery against cancers such as triple negative breast cancers.

The benefits of the filing dates of U.S. provisional application 62/744,233, filed 11 Oct. 2018, and of U.S. provisional application 62/898,266, filed 10 Sep. 2019, are claimed under 35 U.S.C. § 119(e) in the United States, and are claimed under applicable treaties and conventions in all countries.

This invention was made with Government support under NSF EPSCoR grant number 1632854 awarded by the National Science Foundation. The United States Government has certain rights in this invention.

### TECHNICAL FIELD

This invention pertains to biopolymers, to nanoparticles made from those biopolymers, and to delivery of compounds such as drug compounds using those nanoparticles.

### BACKGROUND ART

There is an unfilled need for bio-friendly materials with controlled properties and degradation profiles. Biomaterials synthesized or assembled from combinations of natural and synthetic polymers can have a wide range of characteristics.

Synthetic polymers such as the polyester poly(lactic-co-glycolic acid) (PLGA) are widely used in biopharmaceutical applications. PLGA is biocompatible and biodegradable. This hydrophobic linear co-polymer is usually prepared by a ring-opening polymerization of lactic acid and glycolic acid. It is available in various molecular weights and lactic acid:glycolic acid ratios.

There is an unfilled need for bio-friendly materials with controlled properties and degradation profiles for various purposes, including for example drug delivery. Ideally, the materials should be inexpensive, biodegradable, biocompatible, and made from abundant starting materials. Natural polymers such as cellulose, lignin, chitosan, alginic acid, zein, and various polysaccharides have been used to make nanoparticles, microparticles, films, gels, and other delivery platforms. Alternatively, synthetic polymers such as poly(caprolactone) (PCA), poly(D,L lactic-co-glycolic acid) (PLGA), and poly(ethylene glycol) (PEG) have been used, alone or in various combinations, to produce bio-friendly materials useful, e.g., for drug delivery.

PLGA nanoparticles have previously been made with the assistance of surfactants (viz., anionic, cationic, nonionic, or zwitterionic surfactants). PLGA nanoparticles have a hydrophobic core capable of entrapping hydrophobic compounds such as hydrophobic drugs; and the surfactant improves particle stability in aqueous suspension. A drawback is that excess surfactant must be removed (e.g., by ultrafiltration, dialysis, or ultracentrifugation), thus adding cost to the synthesis. To our knowledge, no previous process for making stable PLGA nanoparticles has avoided employing a surfactant.

Lignin (LGN) is an abundant, low-cost, underused naturally-occurring biopolymer. LGN is heterogeneous, hydrophilic, and crosslinked; it is rich in aromatic units; and it has abundant hydroxyl groups that can functionalize with other reagents. Lignin is an irregular polymer formed by a more-or-less random combination of synapyl, coumaryl and coniferyl alcohols, linked by ether links or condensed C—C bonds. Lignin is available commercially as a byproduct of the paper and biofuel industries. Lignin has previously found only limited industrial uses.

It is estimated that 1 in 8 women in the United States will develop invasive breast cancer during their lives. In 2019, it is predicted that 268,600 women in the U.S. will be diagnosed with invasive breast cancer, and that 41,760 will die from it. Rates are comparable in other countries. About 10-15% of all breast cancers are “triple negative” breast cancers (TNBC), meaning that they lack estrogen receptors, progesterone receptors, and human epidermal growth factor receptors. These triple negative cancers typically have a poorer prognosis than hormone receptor-positive breast cancers, particularly during the first five years after diagnosis. Hormone receptor-positive cancers can be targeted through the hormone receptors, while the hormone receptor-negative cancers cannot. The most widely used drug to treat breast cancer is the targeted therapy tamoxifen, which is often considered a “wonder drug” because of its efficacy and low incidence of serious side effects. However, there can be off-target effects from prolonged tamoxifen use, including an increased risk for endometrial cancer. Tamoxifen cannot currently be used to treat TNBC, because the cancer cells lack an estrogen receptor. Patients with TNBC typically present with high-grade disease, and they often experience early recurrence. Because TNBCs lack drug-targetable hormone receptors, they are typically treated with adjuvant therapy rather than an endocrine/targeted therapy. However, endocrine/targeted therapies are usually better tolerated by patients. “Adjuvant” therapies include those such as local irradiation or chemotherapy. Adjuvant therapy is typically administered after primary surgery, which involves lumpectomy followed by whole breast irradiation or mastectomy. The goal is for the adjuvant therapy to eradicate cells that were missed by the surgical resection, or that were undetectable due to micrometastasis. However, chemotherapy often induces comorbidities such as peripheral neuropathy, febrile neutropenia, and cardiovascular disease. Such morbidities, together with the physical and emotional pain from mastectomy, leave much to be desired in the way TNBC is treated. There is an unfilled need for improved targeted therapies for TNBC, and for improved drug delivery vehicles for TNBC.

Unlike radiation and chemotherapy, targeted therapies selectively act on specific molecular targets, ideally attacking cancer cells while sparing normal tissues and having minimal cytotoxic effects on non-target cells. Examples of targeted therapies include hormone therapies, signal transduction inhibitors, gene expression modulators, apoptosis inducers, angiogenesis inhibitors, immunotherapies, and toxin delivery molecules.

One targeted cancer therapy is the MEK1/2 (mitogen-activated protein kinase (MAPK)/extracellular signal-regulated kinase (ERK) inhibitor GDC-0623, which has successfully completed phase I clinical trials in patients with advanced solid tumors. MEK is part of the RAS-RAF-MEK-MAPK/ERK signaling cascade. It activates MAPK/ERK1/2 via phosphorylation. In this pathway, RAS initiates growth-factor mediated signal transduction across the cell membrane by assembling transient signaling complexes that induce transcription, altered cell morphology, migration, cell survival, proliferation, and even senescence. Roughly 20% of human tumors have mutations that activate RAS genes, making RAS a prime target for intervention. However, a successful RAS inhibitor has been elusive. The compound GDC-0623 is a promising inhibitor of the RAS-RAF-MEK-MAPK/ERK signaling cascade. GDC-0623 targets MEK1/2 through phosphorylation inhibition of ERK1/2. GDC-0623 inhibits phosphorylation of ERK1/2, which leads to up-regulation of CDH1 and down-regulation of FRA1, VEGF, and VIM. In breast cancers, the risk of metastasis and the risk of tamoxifen resistance increase following activation of the RAS-RAF-MEK-MAPK/ERK signaling cascade. Also, a more metastatic phenotype, which tends to be more aggressive and deadly, has been correlated with increased expression of the RAS isoform KRAS25. In recent Phase I trials, GDC-0623 was well-tolerated, and showed dose-proportional and time-dependent pharmacokinetic characteristics. There was no significant accumulation of GDC-0623 at steady-state with daily oral dosing, due to its short metabolic half-life.

Unfortunately, promising experimental compounds often fail in clinical trials. It has been suggested that only about 10% of compounds in cancer therapy phase I trials are likely ever to receive FDA approval. The reasons for these failures include low water solubility, low retention in circulating blood, inability to selectively target tumor cells, and lack of tissue penetration. Targeted therapies can sometimes show low efficacy when the drug's target is internal to the cell, and the drug molecule is not readily taken up by the cell. Nanoparticle drug delivery systems (NPDDSs) can help overcome some of these hurdles by facilitating the controlled release of drugs, by active targeting via attached ligands, by passive targeting through enhanced permeation and retention (ERP) effects, and by other avenues to overcome pharmacokinetic limitations associated with conventional drug delivery formulations. NPDDSs selectively deliver chemical payloads to tumor cells, and help mitigate the effects of cancer-fighting agents on the rest of the body. Drug delivery to the tumor by active targeting can be enhanced by passive EPR effects. Passive targeting can, for example, exploit the leaky vasculature that is commonly associated with tumors. NPs in the range of 20-200 nm can exit the leaky vasculature into the tumor, and can also accumulate in the tumor interstitial spaces. Once inside a tumor, NPs tend to retained, because the poorly-formed tumor vasculature is inefficient at removing them. EPR allows NPs to localize in a tumor and then to release their chemical payload. NPDDSs employing active targeting first gain entry to the tumor through passive targeting. Once inside the tumor, ligands on the NPs, such as monoclonal antibodies or antibody fragments, can bind cancer cells for specific targeting. An example of active targeting is to covalently attach target-specific ligands to nanoparticles, such as PE38KDEL-loaded anti-HER2 poly (lactic-co-glycolic acid) nanoparticles that bind to and internalize in HER2-overexpressing breast cancer cells.

Lignin has been conjugated to NPDDSs. For example, alkaline-lignin (AL) has been conjugated to folic acid-polyethylene glycol (FA-PEG), and loaded with the targeted cancer therapy hydroxyl camptothecin (HCPT), to form FA-PEG-AL/HCPT NPs41. As compared to free HCPT, the FA-PEG-AL/HCPT particles were reported to have a 7-fold increase in blood circulation time, and a 5-fold increase in cellular uptake. See K. Liu et al., “Development of novel lignin-based targeted polymeric nanoparticle platform for efficient delivery of anticancer drugs,” ACS Biomaterials Sci. Eng. (2018).

U.S. patent application publication no. 2014/0080992 discloses the graft co-polymerization of lignin and poly (lactic acid). The synthesis employed a metal-free and solvent-free ring-opening polymerization of lactides in presence of lignin to provide lignin-g-PLA polymers. The size and molecular weight were controlled by changing the lignin/lactide ratio and pre-acylation of lignin

A. Kamil et al., “Bioavailability and biodistribution of nanodelivered lutein,” Food Chem., vol. 192, pp. 915-923 (2016) discloses the use of PLGA nanoparticles to enhance lutein bioavailability in vivo in rats.

A. Richter et al., “Synthesis and characterization of biodegradable lignin nanoparticles with tunable surface properties,” Langmuir, vol. 32, pp. 6468-6477 (2016) discloses the preparation of lignin nanoparticles, and the tuning of their surface properties by coating with a cationic polyelectrolyte, poly(diallyldimethylammonium chloride).

I. Gilca et al., “Preparation of lignin nanoparticles by chemical modification,” Iran. Polym. J., vol. 23, pp. 355-363 (2014) discloses a chemical method to modify lignin by hydroxymethylation to obtain nanoparticles.

A. Myint et al., “One pot synthesis of environmentally friendly lignin nanoparticles . . . ,” Green Chem., vol. 18, pp. 2129-2146 (2016) discloses methods for making lignin nanoparticles in DMF solvent with CO₂ antisolvent, and the effects of varying process parameters such as temperature, pressure, solution flow rate on such things as yields, morphology, size, size distribution, and surface area.

M. Merchant et al., “Combined MEK and ERK inhibition overcomes therapy-mediated pathway reactivation in RAS mutant tumors,” PLOS One 12(10): e0185862 (2017) discloses the anti-cancer compounds GDC-0994 and GDC-0623.

R. Panday et al., “Amphiphilic core-shell nanoparticles: Synthesis, biophysical properties and applications (2018) provides a review of the literature on amphiphilic core-shell particles, their synthesis, properties, and uses with an emphasis on drug delivery and gene co-delivery.

### DISCLOSURE OF INVENTION

We have discovered a lignin-based, biodegradable and biocompatible, polymeric nanodelivery composition, one that may be synthesized without any required surfactants. The novel amphiphilic biopolymers have been synthesized by grafting the hydrophilic natural polymer lignin onto the hydrophobic synthetic polymer PLGA to form graft polymers. The graft polymer molecules can then be further assembled into polymeric nanoparticles. The nanoparticles have a typical diameter of 75 nm, which can be higher or lower depending on the particular composition. The nanoparticles may be formed either by self-assembly or by an emulsion-evaporation process. In neither case is surfactant required. In an aqueous environment, the hydrophobic PLGA tends to associate in the core of the nanoparticles, and the hydrophilic lignin tends to associate on the surface of the nanoparticles.

The synthetic polyester poly(lactic-co-glycolic acid) (PLGA) is biocompatible and biodegradable, which are desirable qualities for biopharmaceutical and other uses. By covalently linking the hydrophilic natural polymer lignin with the hydrophobic synthetic polymer PLGA, a new biopolymer with improved properties is obtained. The LGN-PLGA biopolymer can be used, for example, for biopharmaceutical applications, such as LGN-PLGA nanoparticles for drug delivery. Different embodiments are possible. For example, lignin can be obtained commercially as alkaline lignin or as sodium lignosulfonate (both of which are more hydrophilic than native lignin). Either form may be used in practicing this invention. The abundance of lignin hydroxyl groups offers the option of adding other functional groups, if desired, to impart selected properties to the biopolymer. Also, PLGA with various L:G monomer ratios (more or less glycolic acid relative to lactic acid) and molecular weights can be used, to form biopolymers with different properties. With these variations, the newly synthesized biopolymers have “tunable” properties such as degradation profiles, viscoelastic properties, interface characteristics, and adsorption/desorption properties.

In one embodiment, the invention provides improved drug delivery for triple negative breast cancers. For example, the agent GDC-0623 is a promising cancer drug, but the unmodified compound has poor bioavailability. Our novel nanoparticle system, lignin-graft-poly(lactic-co-glycolic acid) (PLGA) NPDDS, loaded with GDC-0623, more efficiently delivers the compound to cells, leading to improved activity against breast cancer. The novel targeted therapies and drug delivery systems assist in battling TNBC, while reducing deleterious effects and comorbidities.

In some embodiments, a lignin-PLGA graft biopolymer has been synthesized with either of two types of lignin: alkaline lignin (ALGN) or sodium lignosulfonate (SLGN), at different LGN:PLGA ratios (e.g., 1:2, 1:4, and 1:6 w:w). H-NMR and FTIR confirmed the conjugation of PLGA to LGN. The (A/S)LGN-PLGA biopolymers have been used to form nanodelivery systems suitable for entrapment and delivery of drugs. The LGN-PLGA NPs were generally small (<200 nm), monodisperse (PDI<0.4), and negatively charged (−48 to −60 mV). The SLGN-PLGA nanoparticles and the SLGN-PLGA nanoparticles had similar size, zeta potential, and PDI. Small-Angle Scattering (SAS) data showed particles with a relatively smooth surface, a compact spherical structure, and a distinct core and shell. The core size and shell thickness varied with the LGN:PLGA ratio. At a 1:6 LGN:PLGA ratio we observed the core-shell structure to change, with the particles perhaps having a more complex internal structure.

As one example, an alkaline lignin-conjugated-PLGA (AL-PLGA) biopolymer nanoparticle drug delivery system (NPDDS) in accordance with the present invention was found to improve the efficacy of the targeted therapy drug GDC-0623 against the TNBC cell line MDA-MB-231, as compared to results using nanoparticles of the more commonly-used PLGA (only). Our AL-PLGA-GDC NPs had an average diameter of 78±4 nm, while PLGA-GDC NPs had a diameter of 201±2.5 nm, making the novel AL-PLGA-GDC better suited for passive targeting, with an enhanced permeability and retention effect. Although one might expect the greater surface area-to-volume ratio could potentially allow premature diffusion (release) of the payload compound, the loaded AL-PLGA NPs surprisingly showed a more sustained release of drug over 48 hours than did the larger NPs. QCM-D modeling showed the AL-PLGA NPs interacting with the lipid bilayer of cells. We confirmed cellular NP uptake by fluorescence microscopy. Cells treated with AL-PLGA-GDC NPs showed a substantial reduction in cell numbers (nearly 50%), as compared to those treated with free GDC-0623 or with PLGA-GDC-0623 NPs. Western blot confirmed that AL-PLGA-GDC-0623 was better at inhibiting phosphorylation of the target protein ERK1/2 than either free GDC-0623 or PLGA-GDC-0623 NPs. We hypothesize that the increased efficiency was due in large part to the NPs' interactions with cell membranes, leading to greater uptake by cells, and to higher local drug concentrations within target cells. Cells treated with GDC-0623 exhibited a different morphology than did untreated cells. Quantitative PCR (qPCR) confirmed that GDC-0623 reversed epithelial-to-mesenchymal transition (EMT) in MDA-MB-231 cells. AL-PLGA-GDC was more effective at reversing EMT than either free GDC-0623 or PLGA-GDC NPs. In multiple assays and measurements, our findings confirmed that lignin conjugation improved the efficacy of PLGA-based nanoparticles in treating cancer cells with the drug GDC-0623.

The LGN-PLGA nanoparticles were synthesized from both alkaline lignin and sodium lignosulfonate without surfactants, providing savings in cost and time as compared to prior, surfactant-based syntheses of PLGA nanoparticles. Previous syntheses of PLGA-containing nanoparticles have used surfactants to stabilize the hydrophobic core (polymer and drugs) in an aqueous base suspension; and excess surfactant is then removed in a purification step (e.g., by ultrafiltration, dialysis, or ultracentrifugation). Also, the new PLGA-lignin biopolymer has minimal nanoparticle aggregation during freeze-drying, meaning that after freeze-drying the LGN-PLGA nanoparticles can be re-suspended in water or in aqueous buffer without significant change in particle size. The NP powder is compact and easy to handle. A higher ratio of PLGA increases average nanoparticle diameter. The LGN-PLGA NPs can be made in a wide range of sizes, surface charges (e.g., with the optional addition of a surfactant or the optional addition of surface functional groups), and loading capacities. They are stable, allowing long storage times, especially when lyophilized. Hydrophobic or hydrophilic drugs can be delivered with the novel nanoparticles, by entrapping them either in the PLGA hydrophobic inner core, or in the lignin hydrophilic outer shell, or both. The particles regulate the release of the entrapped drugs over time. The degradation of the particles is a function of the LGN:PLGA ratio, as well as the MW of the PLGA, and the L:G ratio selected. Other uses for the novel biopolymers include sutures, scaffolds, films, and packaging.

PLGA is a preferred polyester for use in this invention, particularly for drug delivery applications. For some uses, such as agricultural applications, PLGA may be too expensive. For cost-sensitive uses, alternative polyesters such as polyhydroxyalkanoates (PHAs) may be used in place of PLGA.

Table of Abbreviations

ACN: acetonitrile
AL: alkaline lignin
ALGN: alkaline lignin
ALN: alkaline lignin
AL-PLGA: alkaline lignin-conjugated-PLGA
AL-PLGA-GDC NPs: Alkaline lignin-conjugated-PLGA nanoparticles with
GDC-0623 entrapped
AL-PLGA-TRITC NPs: alkaline lignin conjugated PLGA nanoparticles
with TRITC covalently attached
DAD: diode array detector
DCM: dichloromethane
DI water: deionized water
DLS: dynamic light scattering
DSC: differential scanning calorimetry
DMEM: Dulbecco's modified Eagle's medium
DMF: dimethylformamide
DMSO: dimethylsulfoxide
EE: entrapment efficiency
EMT: epithelial-to-mesenchymal transition
ERK: extracellular signal-regulated kinase
EPR: enhanced permeation and retention
FA-PEG: folic acid-polyethylene glycol
HCPT: hydroxyl camptothecin
HPLC: high performance liquid chromatography
LGN: lignin
LNP: lignin-based nanoparticle
MAPK: mitogen-activated protein kinase
MEK1/2: MAPK/ERK
MW: molecular weight
NP: nanoparticle
NPDDS: Nanoparticle drug delivery systems
PCA: poly(caprolactone)
PCR: polymerase chain reaction)
PDI: polydispersity index
PEG: poly(ethylene glycol)
PHA: polyhydroxyalkanoate
PLGA: poly(lactic-co-glycolic acid) or poly(D,L lactic-co-glycolic acid)
PLGA-GDC NPs: PLGA nanoparticles with GDC-0623 entrapped
PLGA-TRITC NPs: PLGA nanoparticles with TRITC covalently attached
PVA: polyvinyl alcohol
qPCR: quantitative FOR (polymerase chain reaction)
SANS: small-angle neutron scattering
SAXS: small-angle x-ray scattering
SLN: sodium lignosulfate
SLGN: sodium lignosulfate
TEM: transmission electron microscopy
Tg: glass transition temperature
TGA: thermo-gravimetric analysis
TNBC: triple negative breast cancer
TRITC: tetramethylrhodamine isothiocyanate

### MODES FOR CARRYING OUT THE INVENTION

PLGA is more hydrophobic than lignin. Previously, stable PLGA nanoparticles have only been made with the assistance of surfactants. By contrast, the novel approach does not require surfactant, and preferably omits surfactant. Instead, PLGA is covalently linked to a hydrophilic lignin (e.g., alkaline lignin or sodium lignosulfonate) to obtain a new biopolymer with amphiphilic behavior. The amphiphilic property of the new biopolymer allows synthesis of polymeric nanoparticles without the need for surfactants for stabilization. A purification step by dialysis, ultrafiltration, or ultracentrifugation—which had typically been required to remove excess surfactant in prior processes—is not required, thus reducing the time and cost of the nanoparticle synthesis. (Excess surfactant in the final product is undesirable, as it could potentially induce undesirable side-effects and even toxicity.)

Two forms of lignin have been used in the syntheses of prototype embodiments of the invention: alkaline lignin and sodium lignosulfonate. Both forms are commercially available. PLGA (50:50 copolymer) with molecular weight 38-54 KDa was used in the prototype syntheses. PLGA was covalently linked to lignin in a two-step reaction: PLGA was activated (acylated) with oxalyl chloride, and the activated PLGA was then coupled with alkaline lignin or sodium lignosulfonate. The LGN-PLGA biopolymer could then be used to form nanodelivery systems suitable for entrapment and delivery of molecules such as drug molecules. For example, we tested ALGN:PLGA mass ratios of 1:2, 1:4, 1:6, and 1:8. Following polymer characterization by NMR, FTIR, DSC, and TGA, the LGN-PLGA graft polymer was also formed into nanoparticles. Nanoparticles were characterized by size, size distribution, zeta potential, stability, and morphology by DLS, TEM, and SAXS/SANS. The smallest nanoparticles from one set of reactions measured 78.3±1.1 nm (polydispersity index, PDI, of 0.070±0.009) after re-suspension, and were slightly negatively charged (−31±4.1 mV for a biopolymer mass ratio of 1:2). We also tested sodium lignosulfonate (SLGN)-PLGA biopolymers, synthesized at SLGN-PLGA mass ratios of 1:1, 1:2, and 1:4. The SLGN-PLGA nanoparticles were largely similar to the ALGN-PLGA nanoparticles in size, zeta potential, and PDI.

The novel synthesis does not require a polymerization step per se. Using lignin and PLGA starting materials, the synthetic scheme we employed was a “top-down”synthesis rather than a “bottom-up” synthesis. Lignin (or modified lignin) reacts with PLGA to form a novel biopolymer, via ester linkages between lignin hydroxyl groups and PLGA carboxyl groups. The ratio of the one starting material to the other affects both hydrophobicity and size of the resulting nanoparticles.

Lignin may be used in the form in which it is provided as a paper mill byproduct, without further purification; or it may be purified or modified with additional functional groups. It is preferred that the lignin starting material should be “dry” before the reaction, i.e., that most of the water should be removed, e.g., in a vacuum oven or in a desiccator, because water can interfere with the esterification reaction.

It will be appreciated that, although specific embodiments of this invention have been described herein for purpose of illustration, various modifications may be made without departing from the spirit and scope of the invention.

The following examples are put forth to provide those of ordinary skill in the art with a complete disclosure and description of how the compounds, compositions, and methods claimed herein are made and evaluated. The examples are intended to be exemplary, but not to limit the scope of the invention. Efforts have been made to ensure accuracy with respect to numbers (e.g., amounts, temperature, etc.), but some errors and deviations should be accounted for. Unless indicated otherwise, parts are parts by weight (mass), temperature is measured in the Celsius scale (° C.) or the temperature is ambient temperature, and pressure is at or near one atmosphere.

### Synthesis of Biopolymers and Nanoparticles

### Example 1

Poly (lactic-co-glycolic acid) (PLGA) 50:50 (38 to 54 KDa), was purchased from Sigma-Aldrich. The alkaline lignin and the sodium lignosulfonate were purchased from TCI Inc. The oxalyl chloride, dichloromethane (DCM), dimethyl sulfoxide (DMSO), trehalose, ethyl ether, ethanol, dimethylformamide (DMF), and ethyl acetate were purchased from Fisher Scientific (Hampton, N.H.).

### Example 2: Synthesis of Alkaline Lignin (Algn)-Plga Biopolymer

Commercially-available lignin is provided either from sulfur-based processes that produce sodium lignosulfonate, or from alkaline-based processes as alkali lignin. Both types were used in this study. Alkaline lignin and PLGA were coupled by acylation. See Scheme 1. Briefly, 2 g of PLGA was dissolved in 30 ml of DCM at room temperature in a 3-neck, round bottom flask. A nitrogen flow was connected to a bubbler bottle with 1 M NaOH to neutralize HCl produced during the reaction. After the PLGA had completely dissolved at room temperature, an excess (5 Eq.) of oxalyl chloride was added dropwise at 4° C. with a glass syringe. The reaction mixture was kept at room temperature with mild stirring for 4 hours. The solution was concentrated with a rotovapor (Buchi R-300, Buchi Corporation, New Castle, Del.). The product was precipitated by adding 150 ml ethyl ether, and the white precipitate was washed at least three times to remove impurities. The solids were dried overnight under high vacuum. The second step of the reaction scheme started by dissolving 1 g dry PLGA-CI in 20 ml DMSO. The resulting solution was then added dropwise to a solution of 500 mg ALGN in 20 ml DMSO. The reaction mixture was kept at room temperature overnight under nitrogen flow. The resulting ALGN-PLGA polymer was precipitated by adding 200 ml ethyl ether; and the precipitation step was repeated three times. The precipitated polymer was suspended in 20 ml DCM, and the organic phase was washed with water to remove unreacted lignin to obtain a clear supernatant. The DCM was evaporated with a rotovapor Buchi R-300, and the polymer was dried under high vacuum for 3 days at 30° C. The ALGN-PLGA biopolymer was stored at 2-4° C.

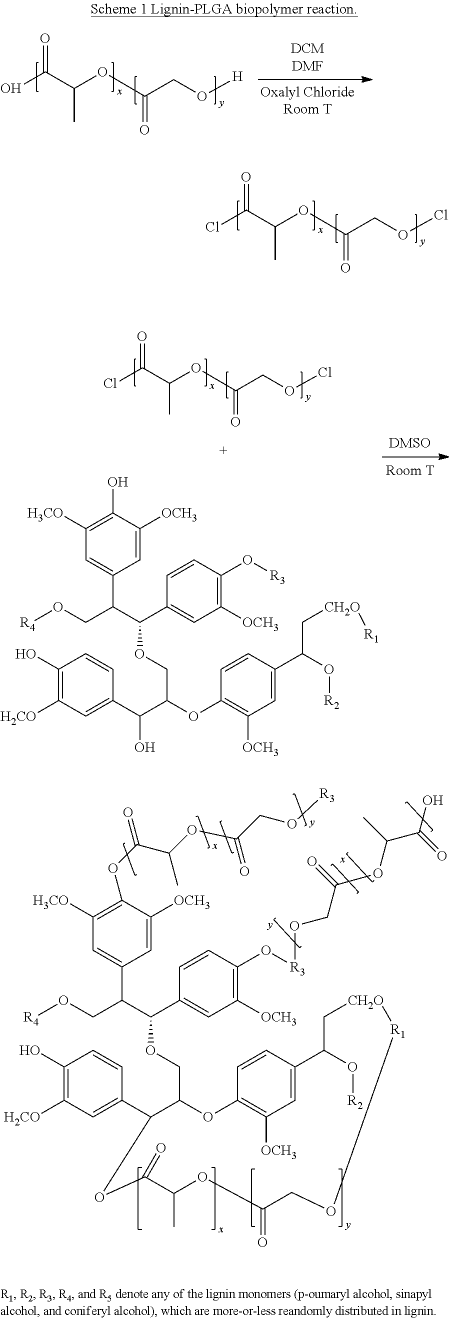

### Example 3: Synthesis of Sodium Lignosulfate (SLGN)-PLGA Biopolymer

Sodium lignosulfate and PLGA were coupled by acylation. The first step, the chlorination of PLGA, was carried out generally as otherwise described in Example 2. The second step was the addition of SLGN (Cat. No. L0082, TCI). Several ratios of SLGN:PLGA were used to study the effect of that ratio on the characteristics of the polymeric nanoparticles; here we outline the procedure for synthesizing the 1:2 ratio as an illustrative example. The dry SLGN (500 mg) was dissolved in 20 mL dimethylsulfoxide at room temperature under mild mixing. PLGA-CI (1 g) was dissolved in 20 mL DMSO with mild stirring, and then slowly added to the SLGN solution. The reaction was maintained at room temperature, and was stopped after 24 h. SLGN-PLGA product was washed with cold water and ether to remove unreacted SLGN. The SLGN-PLGA was dried under high vacuum overnight, and samples were stored at −20° C.

### Example 4: Synthesis of ALGN-PLGA Nanoparticles

Polymeric nanoparticles were synthesized by an emulsion-evaporation technique, with two changes from procedures that have typically been used in other emulsion-evaporation methods: (1) There was no need for surfactants. (2) There was no need for a purification step.

Briefly, 150 to 500 mg PLGA-lignin was dissolved in 5 ml ethyl acetate at room temperature under strong stirring. Next, the solution was added to 50 ml DI water. After 10 minutes' mixing, the suspension was homogenized with a microfluidizer (Microfluidics Corp., Westwood, Mass.) at 30,000 psi (200 MPa), four times, at 4° C. Alternatively, sonication could be used, particularly for small volumes. Next, the organic solvent was evaporated in a rotovapor R-300 (Buchi Corporation, New Castle, Del.) at 32° C. under vacuum for at least 45 min. Finally, trehalose was added (1:1 mass ratio) as a cryoprotectant, and the samples were placed in a freeze-drier (FreeZone 2.5, Labconco Corporation, Kansas City, Mo.) for 2 days at −80° C. to remove water. The resulting lignin-PLGA biopolymer nanoparticles were stored at −20° C. The biopolymeric nanoparticle powder was re-suspended in low conductivity water for characterization.

### Example 5: Synthesis of SLGN-PLGA Nanoparticles

Synthesis of SLGN-PLGA nanoparticles followed a similar procedure. Briefly, 40 mg of the biopolymer was dissolved in 1 mL ethyl acetate at room temperature for 30 min. The organic phase was added drop-wise to 10 ml water in an ice bath with sonication. The emulsion was sonicated (Vibra-Cell VC 750, Sonics & Materials Inc., Newton, Conn.) for 12 min (40% amplitude, 5 s “on” and 2 s “off”). Next, the solvent was evaporated in a rotovapor Buchi R300 (Buchi Corporation, New Castle, Del.) under vacuum. D-(+)-Trehalose was added to protect the nanoparticles during freeze-drying. The clear suspension was freeze-dried (Labconco Corporation, Kansas City, Mo.) for 48 hours, and the resulting powder was stored at −20° C.

### Example 6: Biopolymer Characterization

The ALGN-PLGA and LGN-PLGA conjugates were characterized by H-NMR, (Bruker 500, Billerica, Mass.) at 500 Hz in DMSO. The conjugates were also analyzed by FT-IR with a Bruker Tensor 27 (Bruker, Billerica, Mass.) instrument, with dry samples placed in the detector.

### Example 7: Entrapment of Active Components

For proof of concept that the LGN-PLGA NPs can be effectively used to deliver active components, an experimental breast cancer drug was entrapped in the novel nanoparticles. Briefly, the drug GDC-0623 (an MEK inhibitor; available from Selleckchem or Cayman Chemical) was added in the organic phase (ethyl acetate), following procedures as otherwise described in Examples 4 and 5 to make nanoparticles; the particles were freeze-dried and stored at low temperature.

### Example 8: Nanoparticle Characterization

Size, polydispersity, and zeta potential of the nanoparticles were measured by dynamic light scattering (DLS) (Malvern Zetasizer ZS, Malvern Panalytical, Westborough, Mass.). Briefly, 1 ml of sample at a concentration of 0.2-0.4 mg/ml was placed in a cuvette or a zeta potential cell and analyzed at 25° C. Nanoparticle morphology was analyzed by Transmission Electron Microscopy (TEM) (JEM-1400, Jeol USA Inc., Peabody, Mass.). One droplet of suspension with a contrast agent was placed on a carbon-copper grid, excess sample was removed, and the sample was dried for 10 minutes before TEM images were acquired. The LGN-PLGA conjugates were analyzed by H-NMR (Bruker 500, Billerica, Mass.), FT-IR (Bruker Tensor 27, Bruker, Billerica, Mass.), and TGA.

### Example 9: Release of GDC-0623 from LGN-PLGA Nanoparticles

Release of the test drug GDC-0623 from LGN-PLGA nanoparticles (Nps) was studied at 37° C. in PBS buffer, pH 7.4. The nanoparticle powder was re-suspended in PBS buffer at a concentration of 15 mg/ml, and samples were placed in a dialysis bag (molecular weight cut off 12,000-14,000 Da). The samples were placed in an incubator at 37° C., and 0.2 ml samples were taken from inside the dialysis bags at selected times. The collected samples were mixed with 0.8 ml acetonitrile, and vortexed for 30 min. Samples were stored at 4° C. until analysis. The supernatant was analyzed by HPLC (Agilent series 1200) for the presence of GDC-0623. The HPLC mobile phase was a water: acetonitrile (ACN) mixture, with a gradient starting with 90% water and finishing with 100% ACN after 25 min. The injection volume was 10 μL. All data were collected in triplicate.

### Example 10: NMR and IR Analysis of LGN-PLGA Biopolymers

The ALGN-PLGA biopolymer was analyzed by H-NMR and FT-IR. H-NMR showed chemical shifts at 2.08 to 2.12 ppm, corresponding to the aliphatic and aromatic groups of lignin, as well as typical peaks for PLGA (4.5-4.7, 1.7-1.95). FT-IR showed typical lignin and PLGA bands. Typical vibration bands of lignin were seen at 1590-1596 cm⁻¹ and 1508-1510 cm⁻¹, corresponding to vibrations of the aromatic rings (α-carbonyl groups symmetric and asymmetric, respectively). Also, an absorption band at 1030-1050 cm⁻¹ showed the primary and secondary alcohol groups from alkaline lignin-PLGA and lignosulfonate-PLGA. The PLGA stretching bands from C—CO—O (symmetric and asymmetric) vibrations between 1300 and 1150 cm⁻¹ were seen in all samples.

H-NMR plots for alkaline lignin-PLGA and lignosulfonate-PLGA graft polymers showed characteristic peaks for PLGA at 5.1-5.2 and 4.6-4.8 ppm, corresponding to the lactide (CH groups) and glycolide (CH₂ groups) monomers, respectively. The methyl groups of lactide were seen at approximately 1.5-1.7 ppm. All alkaline lignin and lignosulfonate conjugates at the various PLGA ratios showed similar patterns. The lignin showed aromatic hydrogens at a range of 6.4 to 7.5 ppm. The —OCH₃ peaks at 3.7-3.8 ppm appeared in all conjugates formed with alkaline lignin or lignosulfonate. Also, —OCOCH₃ was observed at 1.7-2.2 ppm in all synthesized graft polymers. Water and DMSO peaks were present in the samples at 3.3-3.4 and 2.65, respectively.

### Example 11: ALGN-PLGA Nanoparticle Characterization

The ALGN-PLGA nanoparticles presented a nearly spherical shape by TEM, with a highly uniform size distribution, even without surfactant in the synthesis. The TEM images showed features consistent with an outer shell (hydrophilic lignin) surrounding an inner core (hydrophobic PLGA). When the biopolymer had a higher lignin concentration (e.g., 1:2 ALGN:PLGA w/w), the nanoparticles were smaller than those formed from biopolymer with less lignin (e.g., 1:8 ALGN:PLGA w/w) (Table 1). The ALGN-PLGA nanoparticle size was not significantly affected when the nanoparticles were suspended in water at pH from 1.6 to 8.6. Zeta potential was affected by the pH, from −5 mV to −35 mV over the pH range studied.

TABLE 1
ALGN-PLGA and SLGN-PLGA polymeric nanoparticles characteristics
based on DLS at pH 6

| | | | Zeta Potential |
|---|---|---|---|
| Polymer (w/w) | Size (nm) ± S.D. | PDI ± S.D. | (mV) ± S.D. |
| 1:2 ALGN-PLGA | 113.8 ± 3.4 | 0.313 ± 0.029 | −53.8 ± 6.9 |
| 1:4 ALGN-PLGA | 144.1 ± 3.0 | 0.351 ± 0.043 | −60.0 ± 0.8 |
| 1:6 ALGN-PLGA | 195.5 ± 2.5 | 0.333 ± 0.006 | −52.2 ± 0.8 |
| 1:2 SLGN-PLGA | 106.5 ± 2.7 | 0.258 ± 0.029 | −48.2 ± 1.8 |
| 1:4 SLGN-PLGA | 120.8 ± 1.4 | 0.282 ± 0.022 | −53.3 ± 1.0 |
| 1:6 SLGN-PLGA | 193.7 ± 3.7 | 0.276 ± 0.040 | −55.8 ± 0.3 |

The mean size for 1:2 and 1:4 LG:PLGA, for both alkaline and lignosulfonate, was not strongly influenced by pH. For the SLG:PLGA 1:6, however, the size decreased from 350 to 290 nm when the pH increased from 2 to 10. The zeta potential of the ALG-PLGA and SLG-PLGA nanoparticles was negative across the pH range tested; for SLG-PLGA nanoparticles it decreased from −40 to −70 mV as pH increased from 2 to 10.

The nanoparticle size can be modified by varying the lignin: PLGA ratio, with the size increasing for higher proportions of PLGA. The ALGN-PLGA nanoparticles synthesized with mass ratios of 1:2 and 1:4 had the smallest mean particle size in our experiments to date, under 100 nm, under 70 nm, and even down to 50 nm, with a narrow size distribution and a negative zeta potential. It has previously been difficult to produce such biocompatible nanoparticles having such a small size with the ability to entrap and deliver drugs and other compounds—e.g., antioxidants, anti-inflammatory agents, anti-cancer compounds, etc. Smaller particles generally enable better cellular uptake of the drug (or other payload). Alternatively, higher (relative) loadings are possible by tuning the particle size. Optionally, particle properties may be altered by the addition of surfactant. The particles are biocompatible and biodegradable; they may be used for various modes of drug delivery, including e.g., oral, parenteral, nasal, ocular, intravenous, intramuscular, suppository, dermal patches, etc. The nanoparticles are stable over a wide range of pH.

### Example 12: Small-Angle Neutron Scattering (SANS)

Freeze-dried ALGN-PLGA 1:2 nanoparticles with cryoprotectant trehalose were re-suspended in D₂O and pre-filtered with 0.45 μm PVDF syringe filters to obtain nanoparticles in solution. Small-Angle Neutron Scattering (SANS) data were obtained with the NG 7 SANS instrument at the NIST Center for Neutron Research (NCNR) at National Institute of Standards and Technology (NIST), and the EQ SANS instrument at the Spallation Neutron Source (SNS) at Oak Ridge National Laboratory (ORNL). D₂O and empty cell measurements were taken separately as background data. All measurements were conducted at 20° C. in standard 2 mm cells. Data reduction used Igor Pro and Mantid platforms with standard protocols.

Small-Angle Neutron Scattering (SANS) was used to obtain further information on the particles, including both a more detailed view of the core-shell structure, and also information on potential agglomeration. One aspect of the SANS study was to investigate whether incorporating trehalose before freeze-drying helped prevent aggregation. We used ALGN-PLGA 1:2 particles with the cryoprotectant trehalose. The freeze-dried particles were re-dissolved in D₂O. Neutron scattering profiles were obtained at two different concentrations, 0.2% and 1.25% w/w. To determine the polymer arrangement within the particles, two SANS models were compared. First was a model that assumed the particles were spherical with a smooth outer surface, and with the LGN and PLGA molecules arranged randomly within, without any distinct separation. Second was a model that also assumed the particles were spherical, but that they had a distinct core-shell structure, with hydrophilic lignin in the shell and hydrophobic PLGA in the core. The observed data better fit the core-shell model.

### Example 13: Small-Angle X-ray Scattering (SAXS)

Freeze-dried ALGN-PLGA and SLGN-PLGA particles were re-suspended in deionized water to obtain 12.5 mg/mL solutions. Small-Angle X-ray Scattering data were obtained at beamline 4-2 of the Stanford Synchrotron Radiation Lightsource (SSRL). An automated sampler system loaded 40 μL aliquots of samples into a capillary cell. Each sample was exposed to the X-ray beam 24 times in 1.0 second exposures as the flow cell gently moved the sample back and forth, to minimize radiation damage from extended exposure. Data were taken at 2 detector distances (3.4 m and 1.1 m) to explore a Q range of 0.003-1 Å⁻¹. Data reduction used standard protocols. Data were analyzed using a modified core-shell model.

SAXS can be used to characterize soft materials such as polymeric nanoparticles at the nano-to-meso scale. It is sensitive to changes in X-ray scattering length densities, which arise from differential interactions of electrons with X-rays within materials. The two different polymeric materials, such as LGN and PLGA, allow SAXS to identify the internal structure of core-shell particles from scattering length densities. SAXS confirmed that the LGN-PLGA particles in the study appeared to possess core-shell structures. The core size and the shell thickness varied with the LGN:PLGA ratios. The surfaces of the particles were relatively smooth. However, the ALGN-PLGA 1:6 and SLGN-PLGA 1:6 particles showed a clear deviation from what would be expected from a spherical core-shell model, suggesting they had a more complex internal structure with possible substructures and irregularities. Hence, particles with the higher PLGA proportions cannot be explained by the same model. The data were best-fit calculated for ALGN-PLGA 1:6 and closest-fitted for SLGN-PLGA 1:6. The polydispersity was fixed to certain values to give the best estimates using a log-normal distribution. The core-shell fit results are given in Table 2.

TABLE 2
SAXS modified core-shell model fit results of ALGN-PLGA and
SLGN-PLGA polymeric nanoparticles

| | Core radius (nm) | Shell thickness | Polydispersity |
|---|---|---|---|
| Polymer (w/w) | ± S.D. | (nm) ± S.D. | % |
| 1:2 ALGN-PLGA | 38.5 ± 1.0 | 2.24 ± 0.1 | 36 |
| 1:4 ALGN-PLGA | 41.2 ± 1.0 | 1.81 ± 0.1 | 36 |
| 1:6 ALGN-PLGA | 126.0 ± NA | 80.0 ± NA | 30 |
| 1:2 SLGN-PLGA | 39.2 ± 1.0 | 20.0 ± 0.3 | 40 |
| 1:4 SLGN-PLGA | 62.0 ± 1.0 | 61.5 ± 1.0 | 30 |
| 1:6 SLGN-PLGA | 91.2 ± 10.0 | 12.7 ± 2.0 | 20 |

### Example 14: Release of Active Ingredient

The nanoparticle suspension with entrapped chemotherapeutic compound GDC-0623 had a light brown color, attributed to the lignin. In PBS, pH 7.4, 37° C., 50% of the drug was released after ˜6 hr.; and nearly 100% after 28 hr.

Preliminary results suggested that the particles can remain in aqueous suspension essentially indefinitely. Due to the particles' very small size, the effects of Brownian (thermal) motion can predominate over the effects of gravity.

### Delivering Nutrients or Other Compounds to Living Plants

### Example 15: Delivering Nutrients or Other Compounds to Living Plants

Nanoparticles in accordance with the present invention may also be used to deliver nutrients or other compounds to living plants. Nanoparticles previously used to deliver compounds to plants have primarily been inorganic. Little previous work has been done with organic nanoparticles in this regard. We examined the effect of the novel, biodegradable, polymeric, lignin-based nanoparticles (LNPs) on soybean plant health. The LNPs were synthesized from lignin covalently linked to poly(lactic-co-glycolic) acid by emulsion evaporation, as otherwise generally described in the previous Examples. The LNPs in this series of experiments carried no payload. Dynamic light scattering showed that the LNPs measured 82±3.1 nm in diameter, with a narrow size distribution. The LNPs had a negative zeta potential of −51±4.3 mV. The LNPs were spherical as viewed by TEM imaging. Soybeans were grown hydroponically, in the presence of three concentrations of LNPs: low (0.02 mg/ml), medium (0.2 mg/ml), and high (2 mg/ml), 28 days after germination. Plants were collected and analyzed after 1, 3, and 7 days of exposure to the nanoparticles. The effects of the LNPs on plant health was assayed by analysis of root length, stem length, chlorophyll concentration, dry biomass of roots and stems, and carbon, nitrogen, and micronutrient absorption. Root length, stem length, and dry biomass did not significantly differ between treatments and controls. Chlorophyll was essentially constant across treatments, and was not significantly different from control. Plants treated with LNPs exhibited increased uptake of aluminum, copper, sodium, and zinc, and decreased uptake of boron and potassium by the roots. Treatments also increased sodium and zinc uptake in the stems. These data support the use of polymeric LNPs as a delivery system in agrochemical applications.

### Drug Delivery System Targeting Triple Negative Breast Cancers

### Example 16: Materials

Dichloromethane (DCM), dimethyl sulfoxide (DMSO), dimethylformamide (DMF), ethyl acetate, ethyl ether, acetonitrile (ACN), oxalyl chloride, and tetramethylrhodamine isothiocyanate (TRITC) were obtained from Fisher Scientifics (Waltham, Mass., USA). Poly(lactic-co glycolic acid) (PLGA) (50:50, 38 to 54 KDa), trifluoroacetic acid, N-Boc-ethylenediamine, and 1-[bis(dimethylamino)methylene]-1H-1,2,3-triazolo[4,5-b]pyridinium 3-oxide hexafluorophosphate) were obtained from Sigma Aldrich (St. Louis, Mo., USA). Alkaline lignin was obtained from TCI chemicals. All compounds were reagent grade.

### Example 17: Synthesis of Lignin-PLGA Biopolymer

The biopolymer synthesis was generally as described in the previous Examples. Briefly: The PLGA chlorination was performed in a three-neck, 500 ml round bottom flask under mild stirring. The reaction was under a nitrogen gas bubbler connected to a wash bottle with sodium hydroxide solution to trap evolved hydrogen chloride. 1 g of PLGA was dissolved in 20 ml dichloromethane (DCM) at room temperature under mild stirring for 20 min. An excess of oxalyl chloride (5 Eq.) was slowly added to the PLGA solution with further addition of DMF. The solution was cooled to 4° C. during the addition. After the addition, the reaction was completed at room temperature over 4 hours. Finally, the polymer was precipitated and washed twice with ethyl ether. The modified PLGA polymer was vacuum-dried overnight.

Several mass ratios of alkaline lignin: PLGA were studied. A mass ratio of alkaline lignin to PLGA of 1 to 2 was selected to entrap the cancer drug. Dry lignin (500 mg) was dissolved in 20 ml dimethyl sulfoxide (DMSO) at room temperature under mild mixing. Next, 1 g of PLGA-CI was dissolved in 20 ml DMSO, which was then slowly added to the lignin solution. The reaction was held at room temperature, and stopped after 24 hrs. The biopolymer lignin-PLGA was washed three times with cold water and ether to remove unreacted lignin. The biopolymer was dried under high vacuum overnight. The synthesized alkaline lignin-PLGA biopolymers were stored at −20° C.

### Example 18: Synthesis of TRITC-Labeled PLGA

We conjugated TRITC to PLGA. First, 2 g of PLGA were dissolved in 30 ml DCM at room temperature; followed by the addition of 104 mg 1-[bis(dimethylamino)methylene]-1H-1,2,3-triazolo[4,5-b]pyridinium 3-oxide hexafluorophosphate), 46.8 mg N-Boc-ethylenediamine, and 0.4 ml N,N-diisopropylethylamine. After 12 hours' stirring at room temperature, the reaction was stopped by adding 150 ml distilled water. Next, the polymer was precipitated by adding 200 ml ethanol, to obtain a white solid. The precipitate was washed twice, and dried under high vacuum overnight. The intermediate product was deprotected by resuspending 1 g of the intermediate product in 20 ml DCM, followed by adding 20 ml trifluoroacetic acid (TFA) (1:1 DMC:TFA v/v). After 35 min at room temperature under mild stirring, the reaction was stopped by precipitation in ethyl ether (200 ml). The activated polymer (PLGA-amine) was dried under high vacuum overnight. The PLGA-amine was then conjugated to TRITC. The PLGA-amine polymer (1000 mg) was dissolved in 30 ml DCM and 0.075 ml triethylamine at room temperature. After 15 min. mixing at room temperature, 25 mg of TRITC were added. The reaction was carried out overnight at room temperature. The organic phase was then washed with water five times in a separatory funnel. The PLGA-TRITC polymer was precipitated by adding ethanol (150 ml) to obtain a dark pink solid. The solid was dried under high vacuum for 2 days, and stored at −20°.

### Example 19: Synthesis of TRITC-Labeled Lignin-PLGA

To synthesize TRITC-alkaline lignin-PLGA, 1 g alkaline lignin-PLGA (AL-PLGA) was dissolved in 30 ml of DCM, and 20 mg of TRITC was added. The reaction was carried out at room temperature for 24 hours. The TRITC-ALPLGA was precipitated in ethyl ether, and then the same protocol was followed as otherwise described in Example 18.

### Example 20: Synthesis of PLGA, PLGA-GDC, and PLGA-TRITC NP

PLGA nanoparticles (PLGA NPs) were used as controls. PLGA nanoparticles with GDC-0623 entrapped (PLGA-GDC NPs), and PLGA nanoparticles with TRITC covalently attached (PLGA-TRITC NPs) were used to visualize cellular uptake. The nanoparticles were synthesized by emulsion/evaporation. In general, 300 mg PLGA polymer was dissolved in 6 ml ethyl acetate. (For the TRITC-containing nanoparticles, 300 mg of a 1:1 mixture of PLGA and PLGA-TRITC was used in the synthesis.) In selected cases, 12 mg of the drug GDC-0623 was added to an organic phase of 25% acetone and 75% ethyl acetate. An aqueous phase was prepared by dissolving 270 mg Tween 80 in 60 ml deionized water. The organic phase was poured into the aqueous phase with stirring. The resulting emulsion was passed through a microfluidizer (Microfluidics, Newton, Mass.) four times at 30,000 psi. Next, a rotary evaporator (Buchi Corporation, New Castle, Del.) was used to evaporate the solvent under vacuum. After evaporation, 6.5 ml of 2% w/v polyvinyl alcohol (PVA) was added, and the mixture was stirred for 20 min. Finally, trehalose was added (1:1 mass ratio of trehalose to nanoparticles) before freeze-drying the nanoparticle samples for 2 days (Labconco, Kansas City, Mo.). The lyophilized samples were stored at 4° C.

### Example 21: Synthesis of AL-PLGA, AL-PLGA-GDC, and AL-PLGA-TRITC NP

The alkaline lignin-conjugated PLGA nanoparticles (AL-PLGA NPs) were used as controls. Alkaline lignin-conjugated PLGA nanoparticles with entrapped GDC-0623 (AL-PLGA-GDC NPs), and alkaline lignin-conjugated PLGA nanoparticles with covalently-attached TRITC (AL-PLGA-TRITC NPs) were synthesized by emulsion/evaporation. The alkaline lignin-PLGA polymer used in the nanoparticle synthesis had a 1:2 mass ratio of alkaline lignin to PLGA. Briefly, 400 mg alkaline lignin-PLGA polymer was dissolved in 6 ml ethyl acetate. Separately, GDC-0623-loaded AL-PLGA nanoparticles (containing 14 mg of the active ingredient GDC-0623) were dissolved in 6 ml ethyl acetate. The synthesis of fluorescent nanoparticles used an organic phase with a 1:3 mass ratio of PLGA-TRITC:AL-PLGA. The mixtures were stirred for 30 minutes at room temperature. The organic phase for the AL-PLGA-GDC NPs was prepared in the same manner as otherwise described above for the PLGA-GDC NPs. The aqueous phase used for all alkaline-lignin-PLGA nanoparticles was 60 mL low conductivity water. The aqueous and organic phases were combined and passed through a microfluidizer (Microfluidics, Newton, Mass.) as otherwise previously described. The solvent was evaporated with a rotovapor (Buchi Corporation, New Castle, Del.) for 1 hour at 35° C. Finally, 450 mg of trehalose was stirred into the aqueous suspension, and the mixture was freeze-dried with a 2.5 L FreeZone lyophilizer (Labconco, Kansas City, Mo.). The lyophilized nanoparticles were stored at 4° C.

### Example 22: NP Size and Other Measurements

Dynamic Light Scattering (DLS) with a Malvern Zetasizer Nano ZS (Malvern Instrument Ltd., Worcestershire, UK) was used to characterize the synthesized nanoparticles in size, polydispersity index (size distribution), and zeta potential.

### Example 23: NP Morphology

Transmission Electron Microscopy (TEM) images were acquired with a JEOL JEM 1400 (Jeol USA Inc., Peabody, Mass.). The polymeric nanoparticles were resuspended in DI water to a final concentration of 2 mg/ml. The sample was mixed with uranyl acetate (2%) (contrast agent) and placed on a carbon grid. Excess was removed from the carbon grid, and the sample was dried for 15 min at room temperature before the grid was placed in the TEM.

### Example 24: Quantification of GDC-0623 by HPLC

GDC-0623 was quantified by high performance liquid chromatography (HPLC) with an Agilent 1200 instrument (Agilent, Santa Clara, Calif.). The column was Zorbax Eclipse XDB C-18 (3×150 mm, 3.5 μm). The mobile phase was acetonitrile (ACN) and water, eluted with a mobile phase gradient. From 0 to 20 min the mobile phase was 5% ACN, from 20 to 21 min it increased to 80% ACN, from 21 to 26 min it increased to 95% ACN, and from 26 to 27 min the ACN concentration decreased back to 5%. The injection volume was 10 μl, and the flow rate was 1 ml/min. The DAD (diode array detector) signal was set at 260 and 280 nm. The elution time was 10.3 min for GDC-0623.

### Example 25: Entrapment Efficiency

Entrapment efficiency was determined by HPLC. A known mass of GDC-0623-loaded particles was dissolved in acetonitrile, and analyzed by HPLC. Entrapment efficiency was given as the percentage of the mass of drug found in the nanoparticles, compared with the mass of the drug initially introduced into the synthesis.

### Example 26: Release Profile of GDC-0623 from PLGA and AL-PLGA NP

PLGA NPs and AL-PLGA NPs were resuspended in 1× PBS at a concentration of 9.8 or 5.9 mg/mL, respectively. The resuspended NPs were dialyzed with 1× PBS with shaking at 37° C. Dialysis tubing (Thermo Fisher Scientific, Waltham, Mass., USA) had a molecular weight cutoff of 12-14 kDa. Samples were collected from the dialysis bags at specified times over 48 hours. The PBS was changed twice daily. Samples collected were dissolved in acetonitrile and analyzed by HPLC.

### Example 27: Cancer Cell Line and Cell Culture

Breast cancer cell line MDA-MB-231 was generously donated by the Burrow Lab at Tulane University School of Medicine. Cells were maintained in 5% CO₂ at 37° C. and cultured in Dulbecco's modified Eagle's medium (DMEM) supplemented with 10% Hyclone Cosmic Calf Serum (HyClone Laboratories, Utah), 50 ng/mL insulin (Sigma-Aldrich, St. Louis, Mo., USA), and 1% MEMAA, NEAA, sodium pyruvate, and antibiotic-antimycotic (Gibco, Dublin, Ireland).

### Example 28: Cell Survival Assay

MDA-MB-231 breast cancer cells were seeded at a density of 5000 cells per well in cell culture medium on 96-well Corning Costar tissue culture plastic plates (Corning Life Sciences, Corning, N.Y., USA). After 24 hours in culture, cells were treated with vehicle control (DMSO), PLGA NPs (negative control), AL-PLGA NPs (negative control), free GDC-0623 (positive control), PLGA-GDC NPs, or AL-PLGA-GDC NPs. Selected wells were treated with 100 nM or 10 nM doses of GDC, in either free or nanoparticle-entrapped form. Following treatment, cells were cultured for 5 days and then stained with crystal violet. Crystal violet was eluted with 33% acetic acid, and absorbance at 570 nm was measured with a BioTek Cytation 3 plate reader (BioTek Instruments Inc, Winooski, Vt., USA). The experiment was carried out with both internal replicates (n=3) and biological replicates (n=3).

### Example 29: Western Blot

MDA-MB-231 breast cancer cells were seeded at a density of 2×10⁶ cells per 10 cm tissue culture treated dish (Corning Life Sciences, Corning, N.Y., USA). After 24 hours, cells were treated with DMSO (vehicle), free GDC, PLGA-GDC NPs, or AL-PLGA-GDC NPs. Cells were treated with a 1 nM dose of GDC in either free or NP-entrapped form, and cultured for 24 hours. All samples were washed with PBS and collected on ice for total protein extraction. Protein was extracted with a lysis cocktail of m-PER supplemented with protease inhibitor (0.1%), and phosphatase inhibitor (0.1%) (Thermo Fisher Scientific, Waltham, Mass., USA). Total protein was loaded on the gel for all samples. Samples were prepared for electrophoresis and run at 150 V on Invitrogen Bolt 4½% Bis-Tris Plus electrophoresis gels (Thermo Fisher Scientific, Waltham, Mass., USA) per the manufacturer's specifications. Protein was transferred to an iBlot 2 Transfer Stack with an Invitrogen iBlot 2 Gel Transfer Device (Thermo Fisher Scientific, Waltham, Mass., USA) per the manufacturer's specifications. Samples were blocked in 3% milk for 1 hour. Primary antibodies were p44/42 MAPK (ERK1/2) and phosphor-p44/42 MAPK (ERK1/2) (Cell Signaling Technology, Danvers, Mass., USA), diluted 1:1000. Secondary antibodies, IR-tagged (LiCor Biosciences, Lincoln, Nebr., USA), were diluted 1:10,000 in 5% BSA-TBST. Western blots were analyzed with a LiCor Odyssey Infrared Imaging System. Biological replicates were n=3.

### Example 30: Cell Morphology

Cells were seeded and treated in the same manner as for the cell survival assay. After staining with crystal violet, the cells were imaged at 20× using Nikon Eclipse Ti2.

### Example 31: Quantitative RT-PCR

MDA-MB-231 cells were seeded in T25 flasks (Corning Life Sciences, Corning, N.Y., USA) at a density of 500,000 cells per flask in culture medium. After 24 hours in culture, cells were treated with vehicle (DMSO), 1 nM free GDC-0623 (positive control), PLGA-GDC NPs, or AL-PLGA-GDC NPs, and were collected 24 hours post-treatment. Total RNA was isolated from cells with a Qiagen RNeasy Kit (Qiagen, Germany). 1 μg total RNA was used to generate cDNA with the qScript cDNA SuperMix (Quantabio, USA) synthesis kit per the manufacturer's protocol. Gene expression analysis was run with a PerfeCTa SYBR® Green SuperMix (Quantabio, USA). Total gene expression analysis was carried out with the ΔΔCt (fold expression) method. Data was normalized versus the ACTIN housekeeping gene with biological triplicates±SEM, n=3.

### Example 32: Cellular Uptake of PLGA and AL-PLGA NP

MDA-MB-231 breast cancer cells were seeded on 35 mm, 1.5 cover glass bottom, cell imaging dishes (MatTek Corporation, Ashland, Mass., USA). Cells were seeded at a density of 45,000 cells/dish, and cultured for 24 hours. TRITC NPs were applied to cells at 1.0 or 0.1 mg/mL for 3 hours. Cells were washed three times with PBS, fixed with 10% PFA, and stained with DAPI according to the manufacturer's protocol, and then imaged with Nikon Eclipse Ti2 with NIS Elements AR software (Nikon Inc., Melville, N.Y., USA). 2×2 binning was used to increase the signal-to noise ratio for TRITC.

### Example 33: Quantification of Cellular Uptake

Uptake of nanoparticles was assayed based on the intensity of fluorescence from NPs inside cells using Fiji software.

### Example 34: Nanoparticle Characterization

Three types of particles were synthesized: TRITC-conjugated fluorescent NPs, empty NPs (negative control), and NPs with entrapped drug. PLGA-TRITC and AL-PLGA-TRITC fluorescent NPs were synthesized for the nanoparticle uptake study. All other particles were synthesized for the in vitro drug study. Since PLGA is an established biomaterial for NPDDSs, PLGA NPs were used as a benchmark for comparing the AL-PLGA-based NPs. The NPs were characterized by average diameter, PDI, zeta potential (ζ), and entrapment efficiency (EE). See Table 3. All three types of PLGA-based NPs shared similar characteristics. However, not all the AL-PLGA based NPs were similar: The AL-PLGA-TRITC yielded much larger NPs than those made from AL-PLGA or AL-PLGA-GDC. The AL-PLGA-TRITC NPs also had a much more negative ζ. Overall, the AL-PLGA based NPs were much smaller than the PLGA-based NPs. The EE of GDC in PLGA NPs was slightly higher than that for AL-PLGA NPs: 74% and 67%, respectively. The smaller AL-PLGA NPs (diameters under 200 nm) have desirable NPDDS characteristics, because they will perform better in passive targeting based on the EPR effect.

TABLE 3
Nanoparticles characterization

| | NPs | Diameter | | Zeta Potential | EE | TRITC μg/mg |
|---|---|---|---|---|---|---|
| NPs sample | description | nm | PDI | (mV) | % | NPs |
| PLGA−TRITC | fluorescent | 222 ± 2.0 | 0.088 ± 0.014 | −36.6 ± 1.4 | nia | 0.5 6 ± 0.011 |
| L−PLGA−TRITC | fluorescent | 169 ± 16 | 0.206 ± 0.018 | −70.1 ± 2.0 | nia | 0.20 ± 0.004 |
| PLGA | empty | 223 ± 1.0 | 0.044 ± 0.004 | −30.4 ± 0.1 | nia | nia |
| L−PLGA | empty | 92 ± 3.7 | 0.089 ± 0.014 | −52.0 ± 3.3 | nia | nia |
| PLGA−GDC | loaded | 201 ± 2.5 | 0.109 ± 0.017 | −37.6 ± 0.9 | 74 ± 1.4 | nia |
| L−PLGA−GDC | loaded | 78 ± 4.0 | 0.091 ± 0.014 | −48.1 ± 3.2 | 67 ± 2.3 | nia |

TABLE 4
ALGN-PLGA and SLGN-PLGA polymeric nanoparticles characteristics
based on DLS at pH 6

| | | | Zeta Potential |
|---|---|---|---|
| Polymer (w/w) | Size (nm) ± S.D. | PDI ± S.D. | (mV) ± S.D. |
| 1:2 ALGN-PLGA | 113.8 ± 3.4 | 0.313 ± 0.029 | −53.8 ± 6.9 |
| 1:4 ALGN-PLGA | 144.1 ± 3.0 | 0.351 ± 0.043 | −60.0 ± 0.8 |
| 1:6 ALGN-PLGA | 195.5 ± 2.5 | 0.333 ± 0.006 | −52.2 ± 0.8 |
| 1:2 SLGN-PLGA | 106.5 ± 2.7 | 0.258 ± 0.029 | −48.2 ± 1.8 |
| 1:4 SLGN-PLGA | 120.8 ± 1.4 | 0.282 ± 0.022 | −53.3 ± 1.0 |
| 1:6 SLGN-PLGA | 193.7 ± 3.7 | 0.276 ± 0.040 | −55.8 ± 0.3 |

The PLGA NPs were much larger than the AL-PLGA-based NPs. The size of the nanoparticles was similar, with or without drug molecule loading. A surfactant layer surrounding the PLGA-based NPs could be seen in the TEM images. The surfactant associated with the PLGA-based NPs presumably inhibits agglomeration of the PLGA NPs. The AL-PLGA NPs (with no surfactant) displayed a core-shell structure visible in TEM, with lignin on the surface and PLGA at the core of the particles. When loaded with GDC-0623, both delivery systems exhibited an initial burst-release, followed by a slower, more sustained release over tens of hours. The AL-PLGA NPs displayed a slower release. The PLGA-GDC NPs and AL-PLGA-GDC NPs had released nearly all drug by 32 and 48 hours, respectively. The slower release of GDC-0623 from the AL-PLGA-GDC NPs was quite surprising, because the smaller NPs had a greater surface-area-to-volume ratio, which would ordinarily be expected facilitate faster diffusion. We propose that the slower release was attributable to the lignin on the surface of the AL-PLGA-GDC NPs.

### Example 35: In Vitro Drug Study

To determine a preferred dose of GDC-0623 to be used against the MDA-MB-231 breast cancer cell line in this study, we screened its effect in vitro at five concentrations from 1 nM to 10 μM. The MDA-MB-231 cell line was used as a model for this study because it expresses a mutation causing upregulation of the KRAS pathway. When we compared the results to DMSO (vehicle), we observed significant reductions in cell numbers at 10 μM, 1 μM, and 100 nM—but not at 10 nM or 1 nM. At 100 nM, a 50% reduction in cell numbers was seen. The 100 nM dose was then chosen as the preferred dose for the next round of experiments. Next, AL-PLGA-GDC NPs were compared against both negative controls (DMSO, PLGA NPs, and AL-PLGA NPs) and positive controls (free GDC-0623 and PLGA-GDC NPs). The PLGA and the AL-PLGA negative controls did not affect the relative numbers of cells, indicating that the NPs themselves were not cytotoxic. Indeed, the number of cells treated with negative NP controls actually grew slightly; we attributed the increase to the trehalose cryoprotectant, which could have provided a minor source of carbohydrate nutrients for the cells.

At both 100 nM and 10 nM, the AL-PLGA-GDC induced a much sharper reduction in cell numbers than either the PLGA-GDC NPs or the free GDC-0623. Indeed, the effect of the PLGA-GDC NPs at these concentrations was not significantly different from control, while AL-PLGA-GDC induced a substantial and significant reduction in cell numbers compared both to control and to PLGA-GDC NPs, at both concentrations. It was quite surprising that the AL-PLGA-GDC NPs improved the efficacy of GDC-0623 at doses below those initially found to be significant in the dosing curve. By contrast, the PLGA-GDC NPs did not enhance efficacy at the lower dose. See Tables 5, 6, and 7.

TABLE 5
Dose response of MDA-MB-231 breast cancer cell line when treated with
DMSO (vehicle) and GDC-0623 concentrations from 10 pM to 1 nM. n = 3,
*p ≤ 0.05, **p ≤ 0.01.

| | Mean Relative Cell | |
|---|---|---|
| Treatment | Number (%) | SEM (%) |
| DMSO | 100.0 | 0.0 |
| 10 μM GDC-0623 | 30.9** | 5.7 |
| 1 μM GDC-0623 | 31.3** | 6.7 |
| 100 nM GDC-0623 | 46.8* | 9.3 |
| 10 nM GDC-0623 | 83.9 | 6.8 |
| 1 nM GDC-0623 | 96.1 | 5.9 |

TABLE 6
MDA-MB-231 cell line treated with DMSO, PLGA NPs (negative control),
AL-PLGA NPs (negative control), free GDC-0623 (positive control), PLGA
NPs with entrapped GDC-0623, and AL-PLGA NPs with entrapped
GDC-0623. n = 3, *p ≤ 0.05, **p ≤ 0.01, ***p ≤ 0.001.

| | Mean Relative Cell | |
|---|---|---|
| Treatment | Number (%) | SEM (%) |
| DMSO | 100.0 | 0.0 |
| PLGA | 107.8* | 1.7 |
| L-PLGA | 110.6 | 3.9 |
| 100 nM free GDC-0623 | 51.4** | 3.7 |
| PLGA-100 nM GDC-0623 | 46.4** | 3.5 |
| L-PLGA-100 nM GDC-0623 | 25.6*** | 1.2 |
| PLGA | 111.0 | 5.1 |
| L-PLGA | 110.4 | 7.0 |
| 10 nM free GDC-0623 | 87.8* | 1.8 |
| PLGA-10 nM GDC-0623 | 81.5* | 2.8 |
| L-PLGA-10 nM GDC-0623 | 47.8** | 3.9 |

TABLE 7
Results of NPs with entrapped drug, normalized versus free drug. Cells
were treated for 5 days and then stained with crystal violet. Crystal violet
was eluted with 33% acetic acid, and absorbance was read at 570 nm, n = 3,
#p ≤ 0.05, ##p ≤ 0.01.

| | Mean Relative Cell | |
|---|---|---|
| Treatment | Number (%) | SEM (%) |
| 100 nM free GDC-0623 | 100.0 | 0.0 |
| PLGA-100 nM GDC-0623 | 91.0 | 9.1 |
| L-PLGA-100 nM GDC-0623 | 50.1# | 2.6 |
| 100 nM free GDC-0623 | 100.0 | 0.0 |
| PLGA-100 nM GDC-0623 | 93.0 | 4.1 |
| L-PLGA-100 nM GDC-0623 | 54.7## | 5.5 |

### Example 36: Western Blots

Western blots were made to determine whether the AL-PLGA-GDC NPs could enhance GDC-0623's phosphorylation inhibition of ERK1/2 as compared to free GDC-0623 or to PLGA-GDC NPs. The concentration of GDC-0623 was reduced to 1 nM for these experiments, because high levels of cell death occurred at higher concentrations, making it difficult to extract sufficient protein for a Western blot. The results of p-ERK1/2 were normalized to control protein RhoGDlα to account for the potential of unequal protein loading. When normalized to DMSO, all treatments elicited a similar response, with AAL-PLGA-GDC NPs performing slightly better at inhibiting phosphorylation of ERK1/2. However, when the results were normalized to free GDC-0623, only AL-PLGA-GDC significantly increased the inhibition of phosphorylation. The Western blot demonstrated that AL-PLGA-GDC has lower p-ERK1/2 activity, that there was no reduction in total ERK1/2 activity, and that there was equal loading of protein as compared to the RhoGDlα control protein.

### Example 37: GDC-0623 Reversed EMT in the MDA-MB-231 Cell Line, an Effect that the Novel Nanoparticles Enhanced

Different populations of the MDA-MB-231 cell line were treated with DMSO (vehicle), free GDC-0623 (positive control), PLGA NPs with entrapped GDC-0623, or AL-PLGA NPs with entrapped GDC-0623. qPCR results for several EMT markers were normalized to DMSO for CDH1, FRA1, VEGF, and VIM. Results were normalized to free GDC-0623. Images were captured to show the resulting morphology of the MDA-MB-231 cell line treated with DMSO, with AL-PLGA NPs (negative control), with free GDC-0623 (positive control), and with AL-PLGA NPs with entrapped GDC-0623. The GDC-0623 concentration was 1 nM for qPCR, and was 100 nM for morphology. The cells treated with GDC-0623 expressed a different morphology as compared to cells treated with negative controls. MBA-MB-231 expressed a more mesenchymal phenotype, and appeared to become more epithelial following treatment. qPCR was conducted to investigate whether GDC-0623 reversed EMT. Upregulation of ERK2 is associated with a more aggressive phenotype in breast cancer cells, presumably by inducing an epithelial-to-mesenchymal transition (EMT). When the qPCR results were normalized to DMSO, only the AL-PLGA-GDC NPs demonstrated a significant increase—indeed, a 15-fold increase—in the expression of epithelial CDH1. Most treatments significantly reduced expression of the EMT markers FRA1, VEGF, and VIM. However, the AL-PLGA NPs induced a larger, and more significant reduction than did the other treatments. When the results were normalized to free GDC-0623, only the AL-PLGA-GDC NPs significantly increased expression of CDH1 compared to free GDC-0623 and PLGA-GDC NPs. Also, AL-PLGA-GDC NPs significantly reduced expression of VEGF and VIM as compared to the PLGA-GDC NPs. The novel NPDDS significantly improved the inhibitory effects of the anti-cancer drug GDC-0623, as compared both to the free drug and the PLGA-entrapped drug. Another observation we made which, to the inventors' knowledge, has not previously been reported, was that GDC-0623 can reverse EMT in the MDA-MB-231 cell line.

### Example 38: AL-PLGA NPs Have Greater Potential for Cellular Uptake than PLGA NPs

We conducted an NP cellular uptake study to test our hypothesis that the AL-PLGA NPs would be efficiently endocytosed to deliver drug inside the cell, likely via enhanced interactions with the cell membrane. TNBC cells were seeded on no. 1.5 glass coverslip dishes, and were then cultured for 24 hours. The cells were either untreated (control) or treated with fluorescent PLGA-TRITC NPs, or with AL-PLGA-TRITC NPs at 0.1 mg/mL for 3 hours. Cells were fixed, stained with DAPI, and imaged at 40×. Cells were imaged with an inverted fluorescence microscope. AL-PLGA NPs showed greater cellular uptake than PLGA NPs. We believe that a major factor in the improved NP uptake was that lignin conjugation produced much smaller particles with different surface properties. Our hypothesis is that this increased uptake is the mechanism underlying the improved efficacy of the AL-PLGA NPDDS as compared to both the free drug and the PLGA-entrapped drug.

### DEFINITIONS/GENERAL DISCUSSION

In discussing the present compounds, compositions, and methods, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention may be described or claimed in a particular statutory class, such as the statutory class of compositions of matter, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

As used in the specification and the claims, the singular forms “a,” “an” and “the” include plural referents unless the context clearly dictates otherwise; and vice versa. Thus, for example, reference to “a functional group,” “an alkyl,” or “a residue” includes mixtures of two or more such functional groups, alkyls, or residues, and the like; and vice versa. Further, a “functional group” or a “group” may consist of just one atom, or it may contain several atoms.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed, and vice versa. It is also understood that each unit between two particular units is also disclosed. For example, if 10 and 15 are disclosed, then 11, 12, 13, and 14 are also disclosed.

References in the specification and claims to parts by weight (or mass) of a particular element or component in a composition denotes the weight (or mass) relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the composition.

A weight (or mass) percent (wt. %) of a component, unless specifically stated to the contrary, is based on the total weight (or mass) of the formulation or composition in which the component is included.

As used herein, the terms “optional” or “optionally” means that the subsequently described event or circumstance or component may or may not occur or be included, and that the description includes both instances where said event or circumstance or component occurs or is included and instances where it does not.

As used herein, the term “subject” or “patient” can be a human or other mammal, including for example a non-human primate, horse, pig, rabbit, dog, sheep, goat, cow, cat, guinea pig or rodent. The term does not denote a particular age or sex. Thus, adult and newborn subjects, as well as fetuses and embryos, whether male or female, are intended to be covered. In one aspect, the subject is a mammal. A “patient” usually refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removing the cause of the associated disease, pathological condition, or disorder. In addition, this term includes palliative treatment, that is, treatment designed for the relief of symptoms with or without curing the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject who is predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician or veterinarian, and been found to have a condition that can be diagnosed or treated by the compounds, compositions, or methods disclosed herein. As used herein, the phrase “identified to be in need of treatment for a disorder,” or the like, refers to selection of a subject based upon need for treatment of the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injections such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “contacting” as used herein refers to bringing a disclosed compound and a cell, target histamine receptor, or other biological entity together in such a manner that the compound can affect the activity of the target (e.g., receptor, cell, etc.), either directly; i.e., by interacting with the target itself, or indirectly; i.e., by interacting with another molecule, co-factor, factor, or protein on which the activity of the target is dependent.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause substantial adverse side effects. The specific therapeutically effective dose level for any particular patient will depend upon a variety of factors including the disorder being treated, the severity of the disorder; the specific composition employed; the age, body weight, general health, sex and diet of the patient; the time of administration; the route of administration; the rate of excretion of the specific compound employed; the duration of the treatment; drugs used in combination or coincidentally with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount,” that is, an amount effective for prevention of a disease or condition.

As used herein, “EC₅₀” is intended to refer to the concentration of a substance (e.g., a compound or a drug) that is required for 50% antagonism of a biological process, or component of a process, including a protein, subunit, organelle, ribonucleoprotein, etc. In one aspect, an EC₅₀ can refer to the concentration of a substance that is required for 50% antagonism in vivo. In a further aspect, EC₅₀ refers to the concentration of antagonist that provokes a response halfway between the baseline and maximum response.

As used herein, “IC₅₀” is intended to refer to the concentration of a substance (e.g., a compound or a drug) that is required for 50% inhibition of a biological process, or component of a process, including a protein, subunit, organelle, ribonucleoprotein, etc. In one aspect, an IC₅₀ can refer to the concentration of a substance that is required for 50% inhibition in vivo. In a further aspect, IC₅₀ refers to the half maximal (50%) inhibitory concentration (IC) of a substance.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein that, based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

In one aspect, the disclosed compounds comprise the products of the synthetic methods described herein. In a further aspect, the disclosed compounds comprise a compound produced by a synthetic method described herein. In a still further aspect, the invention comprises a pharmaceutical composition comprising a therapeutically effective amount of the product of the disclosed methods and a pharmaceutically acceptable carrier. In a still further aspect, the invention comprises a method for manufacturing a medicament comprising combining at least one compound of any of disclosed compounds or at least one product of the disclosed methods with a pharmaceutically acceptable carrier or diluent.

Compositions of the present invention may be administered to deliver pharmaceutical compounds to treat patients (humans and other mammals) with cancer. Thus, the invention includes pharmaceutical compositions containing nanoparticles of the present invention, at least one drug molecule effective against at least one cancer, and a pharmaceutically acceptable carrier. A composition of the invention may further include at least one other therapeutic agent, for example, a combination formulation or combination of differently formulated active agents for use in a combination therapy method.

The present invention also features methods of using or preparing or formulating such pharmaceutical compositions. The pharmaceutical compositions can be prepared using conventional pharmaceutical excipients and compounding techniques known to those skilled in the art of preparing dosage forms. It is anticipated that the compounds of the invention can be administered by oral, parenteral, rectal, topical, or ocular routes, or by inhalation. Preparations may also be designed to provide slow release of the active ingredient. The preparation may be in the form of tablets, capsules, sachets, vials, powders, granules, lozenges, powders for reconstitution, liquid preparations, or suppositories. Preferably, compounds may be administered by intravenous infusion or topical administration, but in some cases may be administered by oral administration.

For oral administration, the compositions can be provided in the form of tablets or capsules, or as a solution, emulsion, or suspension. Tablets for oral use may include the composition mixed with pharmaceutically acceptable excipients such as inert diluents, disintegrating agents, binding agents, lubricating agents, sweetening agents, flavoring agents, coloring agents and preservatives. Suitable inert fillers include sodium and calcium carbonate, sodium and calcium phosphate, lactose, starch, sugar, glucose, methyl cellulose, magnesium stearate, mannitol, sorbitol, and the like; typical liquid oral excipients include ethanol, glycerol, water and the like. Starch, polyvinyl-pyrrolidone, sodium starch glycolate, microcrystalline cellulose, and alginic acid are suitable disintegrating agents. Binding agents may include starch and gelatin. The lubricating agent, if present, will generally be magnesium stearate, stearic acid or talc. If desired, the tablets may be coated with a material such as glyceryl monostearate or glyceryl distearate to delay absorption in the gastrointestinal tract, or may be coated with an enteric coating. Capsules for oral use include hard gelatin capsules in which the active ingredient is mixed with a solid, semi-solid, or liquid diluent, and soft gelatin capsules wherein the active ingredient is mixed with water, an oil such as peanut oil or olive oil, liquid paraffin, a mixture of mono and di-glycerides of short chain fatty acids, polyethylene glycol 400, or propylene glycol.

Liquids for oral administration may be suspensions, solutions, emulsions or syrups or the compound may be provided as a dry product for reconstitution with water or other suitable vehicles before use. Liquid compositions for oral administration may contain pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, which include oils (for example, almond oil or fractionated coconut oil), propylene glycol, ethyl alcohol or water; preservatives (for example, methyl or propyl p-hydroxybenzoate or sorbic acid); wetting agents or emulsifiers such as lecithin; and, if needed, flavoring or coloring agents.

The compositions of this invention may also be administered by non-oral routes. The compositions may be formulated for rectal administration as a suppository. For parenteral use, including intravenous, intramuscular, intraperitoneal, or subcutaneous routes, the compounds of the invention will generally be provided in sterile aqueous solutions or suspensions, buffered to an appropriate pH and isotonicity or in parenterally acceptable oil. They may also be lyophilized prior to administration. Suitable aqueous vehicles include Ringer's solution and isotonic sodium chloride. Such forms will be presented in unit dose form such as ampoules or disposable injection devices, in multi-dose forms such as vials from which the appropriate dose may be withdrawn, or in a solid form or pre-concentrate that can be used to prepare an injectable formulation. Another mode of administration may utilize a patch formulation to affect transdermal delivery. The compositions may also be administered by inhalation, via nasal or oral routes using a spray formulation containing a suitable carrier.

Methods are known in the art for determining effective doses for therapeutic (treatment) and prophylactic (preventative) purposes for the pharmaceutical compositions. The specific dosage level required for any particular patient will depend on a number of factors, including severity of the condition being treated, the route of administration, and the weight of the patient. For therapeutic purposes, “effective dose” or “effective amount” refers to that amount of each active compound or pharmaceutical agent, alone or in combination, that elicits the biological or medicinal response in a tissue, system, animal, or human that is being sought by a researcher, veterinarian, medical doctor, or other clinician, which includes alleviation of the symptoms of the disease or disorder being treated. For prophylactic purposes (i.e., preventing or inhibiting the onset or progression of a disorder), the term “effective dose” or “effective amount” or “therapeutically effective dose” or “therapeutically effective amount” refers to that amount of each active compound or pharmaceutical agent, alone or in combination, that inhibits in a subject the onset or progression of a cancer as being sought by a researcher, veterinarian, medical doctor, or other clinician. Methods of combination therapy include co-administration of a single formulation containing all active agents; essentially contemporaneous administration of more than one formulation; and administration of two or more active agents separately formulated.

### Miscellaneous

As used in the specification and claims, a “therapeutically effective amount” of a composition refers to a quantity of the composition sufficient to be therapeutically effective to prevent, inhibit, slow the progression, or treat the symptoms of a disease, for example a cancer. Depending on context, these measurements and determinations may be statistical in nature, or they may be made in the professional judgment and assessment of an experienced clinician (e.g., a physician or veterinarian experienced in oncology).

The complete disclosures of all references cited in this application are hereby incorporated by reference. Also incorporated by reference are the complete disclosures of the two priority applications, U.S. provisional application 62/744,233, filed 11 Oct. 2018, and U.S. provisional application 62/898,266, filed 10 Sep. 2019. In the event of an otherwise irreconcilable conflict, however, the present specification shall control.

Note that in the interest of brevity, in some instances data and observations are summarized in the present specification that are presented in greater detail in United States provisional priority application 62/898,266, where the more detailed data may be viewed for reference; and the complete disclosure of which is hereby incorporated by reference.

What is claimed:

1. A graft polymer comprising lignin and poly(lactic-co-glycolic acid) (PLGA), wherein said lignin and said PLGA are covalently linked via ester bonds.

2. Core-shell nanoparticles comprising the graft polymer of claim 1, wherein the core predominantly comprises PLGA, and wherein the shell predominantly comprises lignin.

3. A method for making the nanoparticles of claim 2, said method comprising forming an aqueous emulsion of said graft polymer without added surfactant, and evaporating water from the emulsion to leave solid-phase nanoparticles.

4. A drug delivery composition comprising an admixture of the nanoparticles of claim 2 and a drug molecule; wherein said drug molecule associates primarily with said PLGA core if said drug molecule is hydrophobic; wherein said drug molecule associates primarily with said lignin shell if said drug molecule is hydrophilic; and wherein said drug molecule associates with both said PLGA core and said lignin shell if said drug molecule is amphiphilic, containing both hydrophobic and hydrophilic domains.

5. A method for administering a drug molecule to a mammalian patient, said method comprising administering to the patient the drug delivery composition of claim 4; wherein the drug molecule is taken up by the patient's cells in substantially greater amounts than would be taken up from an equimolar amount of the drug molecule alone, otherwise administered to the patient in the same manner, but in the absence of the polymer nanoparticles.

6. The drug delivery composition of claim 4, wherein the drug molecule is GDC-0623.

7. A method for administering GDC-0623 to a mammalian cancer patient, said method comprising administering to the patient the drug delivery composition of claim 6; wherein the GDC-0623 is taken up by the patient's cells in substantially greater amounts than would be taken up from an equimolar amount of GDC-0623 alone, otherwise administered to the patient in the same manner, but in the absence of the polymer nanoparticles.

8. The graft polymer of claim 1, wherein the mass ratio of lignin to PLGA is from 1:1 to 1:6.

9. The graft polymer of claim 1, wherein the lignin comprises an alkaline lignin.

10. The graft polymer of claim 1, wherein the lignin comprises a lignosulfonate.

11. The nanoparticles of claim 2, wherein said nanoparticles have a mean diameter from 50 to 200 nanometers.

12. The nanoparticles of claim 2, wherein said nanoparticles have a mean diameter from 50 to 150 nanometers.

13. The nanoparticles of claim 2, wherein said nanoparticles have a mean diameter from 75 to 100 nanometers.

14. A method of synthesizing the graft polymer of claim 1, said method comprising reacting lignin with PLGA under essentially anhydrous conditions, wherein hydroxy groups from the lignin and carboxyl groups from the PLGA condense to form ester bonds between the lignin and the PLGA.

15. The method of claim 14, wherein at least some carboxylic acid groups from the PLGA are converted to acyl chloride groups before the reaction between PLGA and lignin.

16. A graft polymer comprising lignin and one or more polyhydroxyalkanoates (PHAs), wherein said lignin and said one or more PHAs are covalently linked via ester bonds.

17. Core-shell nanoparticles comprising the graft polymer of claim 16, wherein the core predominantly comprises one or more PHAs, and wherein the shell predominantly comprises lignin.
